(19) United States
(12) Patent Application Publication
(10) Pub. No.: US 2021/0118323 A1
(43) Pub. Date: Apr. 22, 2021

(54) METHOD AND APPARATUS FOR INTERACTIVE MONITORING OF EMOTION DURING TELETHERAPY

(71) Applicant: The Vista Group LLC, Wilmington, DE (US)

(72) Inventor: Roger J. Quy, Scottsdale, AZ (US)

(21) Appl. No.: 17/116,624

(22) Filed: Dec. 9, 2020

Related U.S. Application Data
(63) Continuation-in-part of application No. 14/251,774, filed on Apr. 14, 2014.
Application No. 14/251,774 is a continuation-in-part of application No. 13/151,711, filed on Jun. 2, 2011, now Pat. No. 8,700,009.
(60) Provisional application No. 61/350,651, filed on Jun. 2, 2010.

(51) Int. Cl. G09B 19/00; H04W 4/38; G16H 10/65; G16H 20/70; G16H 50/20; G16H 40/67; G16H 80/00; G06N 20/00; A61B 5/0205; A61B 5/16; A61B 5/00

(52) U.S. Cl. CPC G09B 19/00; A61B 5/02438; G16H 10/65; G16H 20/70; G16H 50/20; G16H 40/67; G16H 80/00; G06N 20/00; A61B 5/02055; A61B 5/165; A61B 5/6898; A61B 5/486; A61B 5/0022; A61B 5/7465; A61B 5/742; A61B 5/0077; A61B 5/4803; H04W 4/38

(57) ABSTRACT

Methods, devices, and systems for monitoring and sharing emotion-related data from one or more users/patients connected via the internet to others or to a remote therapist. An emotion monitoring device (EMD) measures a patient's biometric data obtained from biosensors and computes emotion states relating to emotional arousal and valence. The EMD communicates the emotion data to an internet server via a wireless network. The internet server transmits the emotion data to a remote therapist. The patients' emotion states are shared with the therapist during a teletherapy interaction to compensate for the absence of in-person clinical information. The therapist may also be equipped with an EMD so that the emotion data of the patient and therapist can be compared to derive an objective measure of the therapeutic relationship.

### CROSS-REFERENCE TO RELATED APPLICATIONS

This application is a continuation-in-part of U.S. patent application Ser. No. 14/251,774, filed Apr. 14, 2014, which is a continuation-in-part of U.S. patent application Ser. No. 13/151,711, filed: Jun. 2, 2011, now U.S. Pat. No. 8,700,009, which claims priority to U.S. Provisional Patent Application Ser. No. 61/350,651, filed Jun. 2, 2010, entitled “METHOD AND APPARATUS FOR INTERACTIVE MONITORING OF EMOTION”, the entirety of each being incorporated by reference herein.

### FIELD OF THE INVENTION

The present invention relates to monitoring the emotions of remote patients and therapists using biosensors and sharing such information over the internet.

### BACKGROUND OF THE INVENTION

It is known that human emotional states have underlying physiological correlates reflecting activity of the autonomic nervous system. A variety of physiological signals have been used to detect emotional states. However, it is not easy to use physiological data to monitor emotions accurately because physiological signals are susceptible to artifact, particularly with mobile users, and the relationship between physiological measures and positive or negative emotional states is not straightforward.

A standard model separates emotional states into two axes: arousal (e.g. calm-excited) and valence (negative-positive]. Thus emotions can be broadly categorized into high arousal states, such as fear/anger/frustration (negative valence) and joy/excitement/elation (positive valence); or low arousal states, such as depressed/sad/bored (negative valence) and relaxed/peaceful/blissful (positive valence).

Mental illness creates large costs for society; yet there is a chronic shortage of mental health care providers, particularly in minority and rural communities. The coronavirus disease 2019 (COVID-19) pandemic accelerated the demand and limited access for mental health services. It also engendered a seismic shift to telehealth for remote diagnosis and treatment of patients. The move to health care online resulted in some distinct challenges for teletherapy in mental illness. The lack of in-person presence created significant barriers to effective therapy. Many therapists report difficulty tracking the emotional states of patients, resulting in diminished clinical insights. Empathy—the ability to understand another's state of mind or emotions—is a key component of psychiatry. Therapeutic alliance, also known as working alliance, is a construct that refers to the quality of the collaborative relationship between patient and therapist. Research has shown that therapeutic alliance is a reliable predictor of client efficacy in psychotherapy or counseling, and the most effective therapists are those who focus specifically on building the alliance. It is much more difficult for a therapist to build an empathic working relationship when the patient's emotional responses are unclear during a teletherapy interaction.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY OF THE INVENTION

Systems and methods according to present principles provide ways to monitor emotional states to overcome the lack of in-person presence during remote mental health therapy, e.g. with patients in their home environment.

It is initially noted that a branch of therapy, known as psychophysiology, practices measuring some physiological signals during therapy to inform clinical practice. However, such practice does not incorporate interactive monitoring of emotional valence and arousal responses of remote patients, e.g., in their home environment, nor simultaneously monitoring the emotional responses of the therapist. Thus, one solution to the problem of obtaining effective emotion information during teletherapy is to provide a means to monitor the patient's emotional responses remotely as an alternative source of clinical data.

By monitoring the physiological correlates of emotional states from remote patients and therapists, processing these data with a novel emotion detection algorithm, and sharing the data via the internet, therapists benefit from real-time clinical insights into the mental states of remote patients. Furthermore, by comparing the emotional responses of patients and therapists, a therapeutic alliance indicator enables therapists to form better empathic relationships with their patients. One or more implementations overcome certain disadvantages of the prior art by detecting and monitoring emotional states with mobile devices, such as smart phones, of users in their home environment. In implementations, one or more emotion recognition algorithms derive emotion arousal and valence indices from physiological signals. These emotion-related data are calculated from physiological signals and communicated to and from a software application. The emotion data from multiple persons may be shared in an interactive network. The data maybe encrypted for security and privacy, e.g., compliance with HIPAA regulations. In one implementation, the emotion data are monitored from a patient and a remote therapist to provide real-time clinical feedback to the therapist, and the degree of synchronization of the emotion states of the patient and therapist are compared to determine the therapeutic alliance.

In another implementation, the emotion data monitored from a couple are shared during an online interaction with a therapist or marriage counselor. Emotion ratings can be collected via the internet on user responses to a variety of media, including written content, graphics, photographs, video and music. The stimuli are chosen to reflect issues important to the success of relationships and may be standardized to provide a consistent experience.

This system is designed for mobile use and can be based on a smart mobile device, e.g., iPhone® or Android™ tablet, thus enabling emotions to be monitored in everyday surroundings. Moreover, the system is designed for multiple users that can be connected in an interactive network whereby emotion data can be collected and shared. The use of mobile devices equipped with cellular communications, e.g., the 5G network, to share emotion frees remote clients from the constraints of a home Wi-Fi connection, and may enable a more private location for therapy sessions. Similarly, low Earth orbit (LEO) satellite networks for broadband communications provide increased access to teletherapy for rural populations.

People are often not aware of transient emotional changes so monitoring emotional states can enrich experiences for individuals or groups. Other applications of emotion monitoring include entertainment, such as using emotion data for interactive gaming. Another application is for personal training—for example, learning to control emotions and maintain a healthy mental attitude for stress management, yoga, meditation, sports peak performance and lifestyle or clinical management. In implementations according to present principles, biometric data are processed to obtain metrics for emotional arousal level and/or valence that can provide signals for feedback and interactivity to enhance telepresence between remote users.

Multiple users equipped with emotion monitors can be connected directly, in peer-to-peer networks or via the internet, with shared emotion data. Therapeutic applications include remote cognitive assessment, rehabilitation, and behavioral therapy. For example, seniors suffering from dementia can receive cognitive rehabilitation in their home environment, or patients in long-term recovery programs for addiction disorders can receive remote assessment and precision mental healthcare. Non-therapeutic applications include sharing emotion data to augment video calls for social and business interactions.

In more detail, implementations according to present principles provide systems and methods for interactive monitoring of emotion data by recording one or more physiological signals, in some cases using simultaneous measurements, and processing these signals with an emotion detection algorithm, providing a display of emotion data, and using the data to interact with other users or software. The emotion data can be transmitted to an internet server and shared by more than one user to form an interactive emotion network for applications including teletherapy and social communities, e.g. for virtual group therapy.

Biosensors record physiological signals that relate to changes in emotional states, such as skin conductance, skin temperature, respiration, heart rate, blood volume pulse (BVP), blood oxygenation, electrocardiogram (ECG), electromyogram (EMG), and electroencephalogram (EEG). For a variety of these signals, either wet or dry electrodes are utilized. Alternatively, photoplethysmography (PPG) can be employed, e.g., to record heart pulse rate and BVP. Implantable sensors may also be utilized. The biosensors can be deployed in a variety of forms, including a finger pad, finger cuff, ring, glove, ear clip (e.g., attached to a phone earpiece), wrist-band, chest-band, head-band, hat, or adhesive patch as a means of attaching the biosensors to the subject. The sensors can be integrated into the casing of a mobile phone, game controller, a TV remote, a computer mouse, or other hand-held device; or into a cover that fits onto a hand-held device, e.g., a mobile phone. In other cases, the biosensors may be integrated into an augmented or virtual reality device, e.g., for affective computing.

In some implementations, a plurality of biosensors may simultaneously record physiological signals, and the emotion algorithm may receive these plurality of signals and employ the same in displaying emotion data or responding to the emotion data of other users. In such cases, a plurality of biosensors may be employed to detect and employ emotion signals, or some biosensors may be used for the emotion signal analysis while others are used for other analysis, such as for the detection of motion artifact or the like. Another strategy is to use an array of biosensors in the place of one, which allows for different contact points or those with the strongest signal source to be selected, and others used for artifact detection and active noise cancellation. An accelerometer can be attached to the biosensor to aid monitoring and cancellation of movement artifacts. The signal may be further processed to enhance signal detection and remove artifacts using algorithms based on blind signal separation methods or machine learning techniques. Such signal processing may be particularly useful in cleaning data measured by such biosensors, as user movement can be a significant source of noise and artifacts.

The physiological signals are transmitted to an emotion monitoring device (EMD) either by a direct, wired connection or wireless connection. Short range wireless transmission schemes may be employed, such as a variety of 802.11 protocols (e.g., Wi-Fi), 802.15 protocols (e.g., Bluetooth®), other RF protocols, or other known telecommunication schemes. The EMD can be implemented on a number of devices, such as a mobile phone, tablet computer, smart display, netbook computer, laptop, personal computer, virtual reality headset, or a proprietary hardware appliance. The EMD can be a wearable device, e.g., smart watch or eyewear. The EMD processes the physiological signals to derive and display emotion data, such as arousal and valence components. A variety of apparatus and methods can be used to monitor emotion, typically some measure reflecting activation of the sympathetic nervous system, such as indicated by changes in skin temperature, skin conductance, respiration, heart rate variability, blood volume pulse, or EEG. Deriving emotion valence (e.g., distinguishing between different states of positive and negative emotional arousal) is more complex. Some alternative approaches that can be employed to distinguish between emotional states include the analysis of EMG signals, body heat signatures, voice features, body language, or encoding of facial micro-expressions, (e.g., as monitored by cameras).

Implementations of the invention may employ algorithms to provide a map of both emotional arousal and valence states from physiological data. In one example of an algorithm for deriving emotional states, the arousal and valence components of emotion are calculated from measured changes in skin conductance level (SCL) and changes in heart rate (HR), in particular the beat-to-beat heart rate variability (HRV). Traditionally, valence was thought to be associated with HRV, in particular the ratio of low frequency to high frequency (LF/HF) heart rate activity. By combining the standard LF/HF analysis with an analysis of the absolute range of the HR (max-min over the last few seconds), emotional states can be more accurately detected. By way of illustration, one algorithm is as follows: If LF/HF is low (calibrated for that user) and/or the heart rate range is low (calibrated for that user) this indicates a negative emotional state. If either measurement is high, while the other measurement is in a medium or a high range, this indicates a positive state. A special case is when arousal is low; in this case LF/HF can be low, while if the HR range is high, this still indicates a positive emotional state. The accuracy of the valence algorithm is dependent on detecting and removing artifact to produce a consistent and clean HR signal.

A method of SCL analysis is also employed for deriving emotional arousal. A drop in SCL generally corresponds to a decrease in arousal, but a sharp drop following a spike indicates high, not low, arousal. A momentary SCL spike can indicate a moderately high arousal, but a true high arousal state is a series of spikes, followed by drops. Traditionally this might be seen as an increase, then a decrease, in arousal, but should instead be seen as a constantly high arousal. Indicated arousal level should increase during a series of spikes and drops, so that the most aroused state, such as by anger if in negative valence, requires a sustained increase, or repeated series of increases and decreases in a short period of time, not just a single large increase, no matter the magnitude of the increase. The algorithm can be adapted to utilize BVP as the physiological signal of arousal.

Facial expressions can be encoded to derive emotion data. There is strong evidence that human faces universally express six basic emotions: happiness, surprise, fear, anger, disgust, sadness, plus neutral. The facial region is captured on a camera image, e.g., from a webcam. Facial landmarks and components are identified in the facial region, and various spatial and temporal features are extracted. Facial expressions are determined from these features using pre-trained classifiers. For example, algorithms based on deep learning have been used for feature extraction, classification, and recognition. Techniques, such as facial action unit or neural network mesh models, categorize the facial expressions corresponding to emotions resulting from the physiological activity of facial muscles. However, facial analysis alone may not reliably derive emotion arousal and valence from some subjects who conceal, or do not freely express, their emotions.

Voice analysis is another method that can be used to derive emotion data from features corresponding to underlying physiological changes in voice production, e.g., tightening of the vocal cords. An algorithm extracts voice features from an audio signal, e.g. during a voice or video call. Deep learning, neural networks, statistical, or other known techniques classify these features to obtain emotion data, e.g., arousal, intensity, or anxiety. In addition to the voice features extracted from a video call, various body language features can be identified and extracted for analysis from the video signal, such as posture, head movements, or fidgeting.

The above-described emotion-deriving methods are believed to have certain advantages in certain implementations of the invention. However, other ways of deriving emotion variables may also be employed. As may be seen above, these algorithms generally derive emotion data, which may include deriving values for individual variables such as level of stress. However, they also can generally derive a number of other emotion variables that be thought of as occupying an abstraction layer above a single dimension variable, such as emotional balance (e.g., positivity/negativity), emotional stability (e.g., anxiety/depression), or emotional strength (e.g., resilience/controlling emotions under stress). The emotion-deriving algorithms may be implemented in a software application running in the EMD, or in firmware, e.g., a programmable logic array, read-only memory chips, or other known methods, or running on an internet server.

The system is designed to calibrate automatically each time it is used. Also, baseline data are stored for each user so the algorithm improves automatically as it learns more about each user's biometric data. Accuracy of emotion detection can be improved with the addition of more biometric data—such as skin temperature, respiration, or EEG. Such can either be entered as a module, e.g., as a separate functional input, if an appropriate relationship is known, or could be learned over time by a machine learning algorithm, e.g., using typically unsupervised learning, but also supervised or reinforcement learning.

The emotional arousal and valence data can be expressed in the form of a matrix displaying emotional states. The quadrants in the matrix can be labeled to identify different emotional states depending on the algorithm, e.g., feeling “angry/anxious, happy/excited, sad/bored, relaxed/peaceful”. The data can be further processed to rotate the axes, or to select data subsets, vectors, and other indices such as “approve/disprove”, “like/dislike”, “agree/disagree”, “feel good/feel bad”, “approach/avoidance”, “good mood/bad mood”, “calm/stressed”; or to identify specific emotional states. The emotional states can be validated against standard emotional stimuli (e.g., the International Affective Picture System). In addition, with large data sets, and as noted above, techniques such as machine learning, neural networks, data mining, or statistical analysis can be used to refine the analysis and obtain specific emotional responses. Algorithms based on such techniques can be used to determine the weights and contribution to variance of signals monitored by different biosensors. Known classification methods can be employed to categorize a user's emotional responses to a variety of stimuli so as to provide a comprehensive emotion matrix or profile of the user. The emotion profiles can be sorted and categorized according to external data, e.g., empirical criteria quantifying therapeutic outcomes. For marriage counseling or couple's therapy, the emotion profiles can be evaluated with data quantifying the success of longer-term relationships, as measured between individuals with comparisons of their derived emotion profiles for compatibility. For addiction recovery programs, emotion data can be monitored to derive predictive analytics, e.g., risk of relapse, and personalized treatments. Other implementations may be seen, e.g., for recruiting members to a team, workplace, or organization; or for enhancing the social dynamics of participants in group activities, multiplayer games, negotiations, business discussions, and the like. For example, the interactive network may be used to enhance telepresence in video conferencing between remote participants by monitoring and sharing their emotional responses.

It can be helpful for emotion data to be displayed to the users in graphical form, e.g., arousal and valence values. Other visual or auditory feedback can be utilized, such as a color code or symbol (e.g., “emoticon”) representing the emotional states, e.g., for biofeedback. The biometric and emotion data may be transmitted to an internet server, or a cloud infrastructure, via a wired or wireless telecommunication network. An internet server may send a response back to the user; and with multiple users the emotion data of one user may be transmitted from the server to be displayed on the EMD of other users (assuming appropriate consent and/or anonymization). The server application program stores the emotion data and interacts with the users, sharing emotion data among multiple users in real time or later as required.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE DRAWINGS

FIG. 1 illustrates a general embodiment of an emotion monitoring network according to present principles to determine and share the emotional states of multiple users.

FIG. 2 illustrates monitoring the emotion data of a patient and a therapist during a remote therapeutic interaction.

FIG. 3 illustrates an embodiment of an emotion monitoring device based on a mobile device connected to biosensors and an internet server.

FIG. 4 illustrates a flowchart of a general method for operating an emotion monitoring network.

FIG. 5 illustrates a flowchart of a method to monitor the emotion data of a patient and a therapist during a remote therapeutic interaction.

Like reference numerals refer to like elements throughout. Elements are not to scale unless otherwise indicated.

### DETAILED DESCRIPTION OF THE PREFERRED EMBODIMENTS

Various acronyms are used for clarity herein. Definitions are given below.

The term “subject” as used herein indicates a human subject. The term “user” is generally used to refer to the user of the device or system, which may be synonymous with the subject. The terms “patient” or “client” are used synonymously depending on the application and may also refer to the user and be synonymous with the subject. The term “therapist” may refer to a clinical psychologist, psychiatrist, clinician, behavioral therapist, caregiver, counsellor, facilitator, or other healthcare provider. The term “signal communication” is used to mean any type of connection between components that allows information to be passed from one component to another. This term may be used in a similar fashion as “coupled”, “connected”, “information communication”, “data communication”, etc. The following are examples of signal communication schemes: As for wired techniques, a standard bus or cable may be used if the input/output ports are compatible and an optional adaptor may be employed if they are not. As for wireless techniques, radio frequency (RF) and other such techniques may be used. A variety of known methods and protocols may be employed for short-range, wireless communication including IEEE 802 family protocols, such as Bluetooth®, (also known as 802.15), including Bluetooth Low Energy (BLE), Wi-Fi (802.11), ZigBee™, Wireless USB and other personal area network (PAN) methods, including those being developed. For wide-area wireless telecommunication, a variety of cellular, radio, satellite, optical, or microwave methods may be employed, and a variety of protocols, including IEEE 802 family, wide-area Wi-Fi, Voice over IP (VOIP), LTE, 4G, 5G, and other wide-area network or broadband transmission methods and communication standards being developed. It is understood that the above list is not exhaustive.

Various embodiments of the invention are now described in more detail.

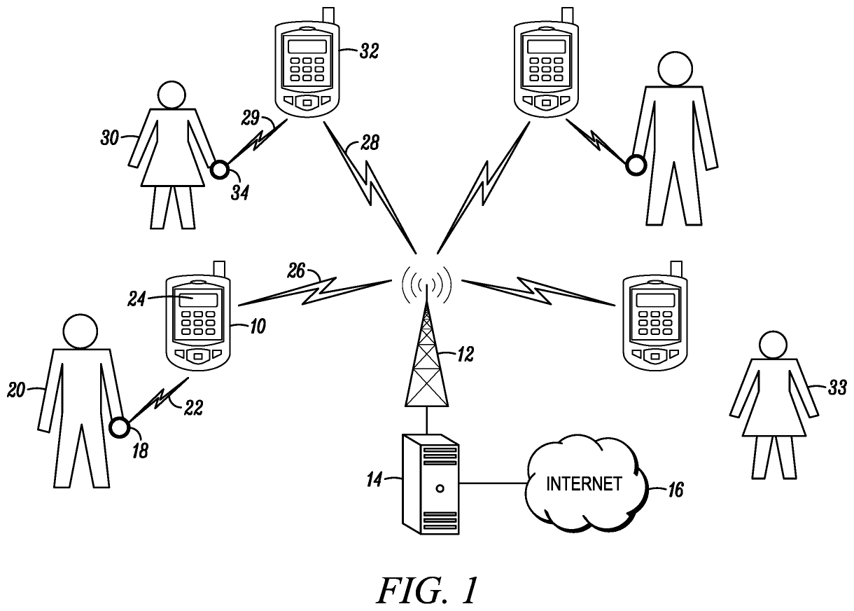

Referring to FIG. 1, a system according to present principles is shown for monitoring emotion (sometimes termed “emotional”) data from two or more subjects connected in a network. It will be understood that in certain implementations the data from just one subject may be employed and processed in accordance with methods described here.

A subject 20 is monitored by one or more biosensors 18 to record physiological signals. The biosensors can be deployed in a variety of forms, including a finger clip, finger cuff, ring, glove, ear clip, wristband, chest-band, eyewear, or headset. Other varieties of biosensors will also be understood; for example, the biosensors may be in the form of a camera to monitor facial expressions, eye movements, or heart rate (e.g., from subtle changes in facial heat signatures, color or movement). Similarly, the biosensor may be in the form of a microphone to monitor voice features or speech data (e.g., semantic content). The physiological signals are transmitted to an emotion monitoring device (EMD) 10, such as a mobile device, e.g., a smart phone or tablet computer, by a wired or short-range wireless connection 22. As described above, EMD 10 further processes the physiological signals and an algorithm derives emotion data from the signals, such as arousal and valence indices. Screen 24 optionally displays emotion data to subject 18.

EMD 10 is connected to a telecommunication network 12 via a wide area, wired or wireless connection 26. The telecommunication network 12 is connected to server 14, including a virtual cloud server that is part of the internet infrastructure 16. EMD 10 transmits the emotion data to an application program running on computer readable media (CRM) in server 14, which receives, processes and responds to the data. The computer readable media in server 14 and elsewhere may be in non-transitory form. A response can be transmitted back to EMD 10. The server 14 also transmits emotion data via connection 28 to be displayed to a remote subject 30. The remote subject 30 is equipped with an EMD 32 and biosensors 34 and may similarly transmit emotion data via connections 29, 28 to the internet server 14. (One remote subject is thus illustrated, but a plurality is similarly equipped, including subject 20.) The server application program stores the emotion data and interacts with the subjects, including receiving, processing, analyzing, and outputting including sharing emotion data among the network of users.

Emotion data may be derived from the signals either using an algorithm operating on the EMD 10 or using an algorithm operating on the server 14, or the two devices may work together to derive emotion data, such as arousal and valence indices. For example, in one implementation, EMD 10 transmits the physiological signals to server 14 and an algorithm on CRM in server 14 derives the emotion data from the signals.

The system of FIG. 1 may be employed for group therapy, e.g. addiction recovery programs. A plurality of subjects 20, 30 is each monitored by one or more biosensors 18, 34, respectively, to record physiological signals, which are transmitted by wired or wireless connections 22, 29 to EMDs 10, 32. The EMD derives emotion data from the physiological signals, and transmits the emotion data to the internet server 14, via wired or wireless connections 26, 28 in a communications network 12, as described above. Server 14 optionally transmits each subject's emotion data by wired or wireless connections 26, 28 to be displayed on EMDs 10, 32. In some implementations, the emotion data are displayed to another member 33 for review. The emotion data for the interaction can be recorded and stored on internet server 14 for asynchronous review later (e.g., by the group, a group facilitator, counselor, therapist, or by a software program or algorithm). It is noted in this regard that member 33 is understood to include not only a group facilitator, counselor, or therapist, but also a virtual therapist, avatar, or conversational artificial intelligence, e.g., AI chatbot application, that responds to the emotion data.

The implementation illustrated in FIG. 1. may be utilized for monitoring and sharing emotion data in other group activities, e.g., monitoring the emotion data of subjects in business meetings, video conferences, or negotiations. In another embodiment, such as in a virtual reality environment, individual emotional data may be employed to control an avatar wherein the emotion data of the subjects are reflected by the avatars, e.g., via facial expressions, colors, symbols, auras, emoticons or the like.

The system of FIG. 1 may be adapted for couples' therapy based on each subject's emotional responses to standardized stimuli. The stimuli are chosen to reflect issues important to the success of relationships, and can include a variety of written content, graphics, photographs, audio or video. Actors portraying couples in various scenarios can be used to explore deeper emotional issues. A display screen 24, which may be incorporated in the EMDs 10, 32, or a separate device (e.g., a desktop computer), displays a series of stimuli to each subject. The stimuli are downloaded to the display from a server 14 connected to the internet 16. Emotion data is monitored, calculated, and displayed for each subject as described above. Thus, the couple can see each other's emotional responses as they converse, which will provide them with insightful information about their relationship. A software application running on the internet server 14 calculates a profile of each subject based on their emotional responses to each stimulus. The application may further use an algorithm to assess the emotional compatibility of the couple utilizing measures from other variables and data sources. For example, the emotion profiles of couples who are happily married can be collected and compared with those who underwent divorce. The algorithm can employ techniques such as statistical methods, machine learning, artificial intelligence, and the like, to draw correlations and contrasts. The emotion data for the interaction is stored on internet server 14 for later review. The emotion data are shared with another member 33, such as a counselor or couples' therapist. It will be understood that each of the stimuli, psychological signals, emotion data, application programs, algorithms, external data sources, or analysis techniques may physically reside on more than one server or different servers (e.g., on a cloud of servers) for storage or multiple processing purposes.

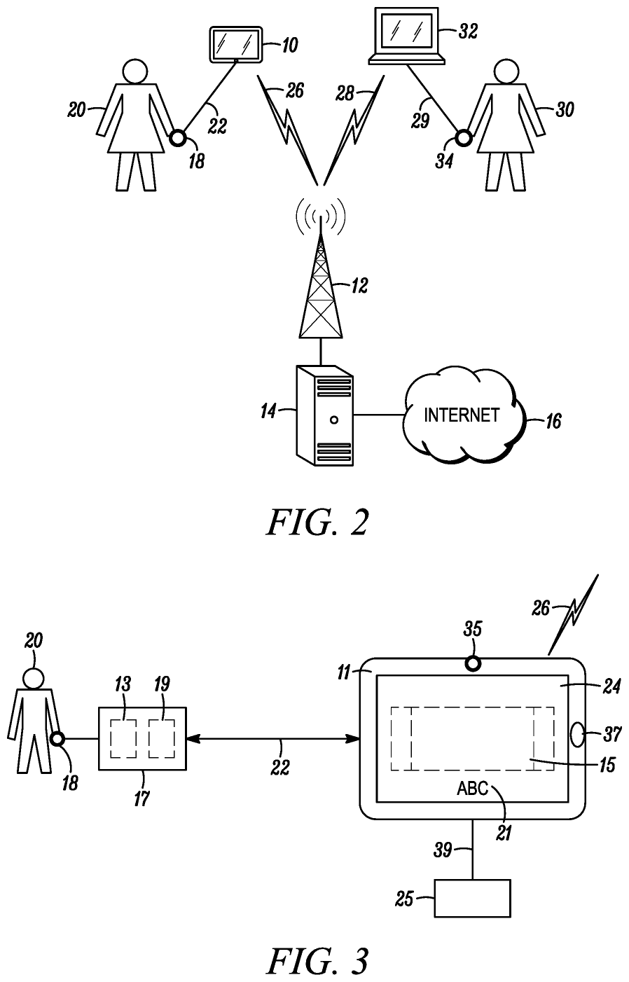

Referring to FIG. 2, an implementation according to present principles is illustrated to monitor and share emotion data to provide metrics of a healthy mental state during teletherapy. A client 20 is monitored by one or more biosensors 18 to record biometric data relating to emotions, which are transmitted to an EMD 10 by a wired or short-range wireless connection 22. The EMD transmits the biometric signals via a wireless connection 26 to an internet server 14. An algorithm on CRM in server 14 derives the emotion data from the biometric signals. The server transmits the emotion data via a similar connection 28 to be displayed to a remote therapist or caregiver 30 on EMD 32, e.g., on a screen depicting a video communication with the client together with a graphical indication of the client's biometric and emotion data. EMD 32 similarly records biometric data relating to emotion from one or more biosensors 34 monitoring the therapist 30, and transmits the biometric data via connection 28, to a server 14 connected to the internet 16. Server 14 in turn calculates the therapist's emotion data. An algorithm on server 14 further processes the biometric and emotion data of the client and of the therapist to derive a measure of therapeutic alliance. The algorithm calculates the degree of synchrony between emotion data employing known signal analysis and statistical techniques. The algorithm is optimized using variance analysis and machine learning techniques together with external data such as self-reports of the therapeutic interaction. Thus the degree of synchrony may not necessarily be a one-to-one mapping of emotion data from patient to therapist but may be an index based on the emotion data of the patient and the emotion data of the therapist as a function of machine learning over time. In some implementations, the algorithms deriving the emotion data and therapeutic alliance may be on a CRM in the therapist's EMD 32. The therapeutic alliance is displayed on the therapist's screen in real-time. The video communication, biometric data, emotion data, and alliance may also be recorded and stored for asynchronous review and analysis. One application of the therapeutic alliance metric is to provide an objective assessment of progress across therapy sessions. Another application is to train therapists or others to enhance empathy and working alliance with clients, especially those from a different racial or cultural background. Optionally, the emotion data of the therapist and/or the therapeutic alliance may be transmitted via connection 26 to be shared with the client. The therapeutic effectiveness can be enhanced by an artificial intelligence program that monitors the emotional responses of the client and guides the therapist according to pre-determined protocols and outcomes data, or that provides guidance according to machine-learned protocols, based or indexed on patients or therapists having similar cultural characteristics, demographics, and so on.

Referring to FIG. 3, an embodiment of EMD 10 is shown based on a web-enabled, mobile device 11, such as an iPhone®, tablet, or smart display, e.g., a video calling device. One or more biosensors 18 measure physiological signals from a subject 20. A variety of types of biosensors may be employed as described above. The biosensors may be integrated into an attachment or casing of the mobile device, for example, camera 35 and microphone 37 are typically integrated in smart mobile devices. An accelerometer 13 optionally may be included to aid detection and removal of movement artifacts.

A short-range wireless transmitter 19, or a direct or wired connection, is employed to transmit the signals from the biosensors via connection 22 to the mobile device 11. An optional adapter 25 connected to the generic input/output port or “dock connector” 39 of the mobile device may be employed to receive the signals. The signals from the biosensors are amplified and processed to reduce artifact in a signal processing unit (SPU) 17, which may be incorporated with the biosensors or implemented in the mobile device. An application program 15 is preloaded or downloaded from an internet server to a CRM in the mobile device. The application program receives and processes the signals from the biosensors. The application program includes a user interface to display information on screen 24, and for the subject to manually enter information by means of a keyboard, buttons or touch screen 21. As illustrated in FIG. 1, mobile device 11 is in data communication with an internet server and transmits signals from the biosensors via wireless connection 26 to the internet server and may also receive emotion data of other users. An algorithm operated on the internet server derives emotion data from the biometric signals, as previously described. Alternatively, application 15 on mobile device 11 includes an algorithm to derive emotion data, or the emotion-deriving algorithms may be implemented in firmware, in which case the application program receives and displays the emotion data.

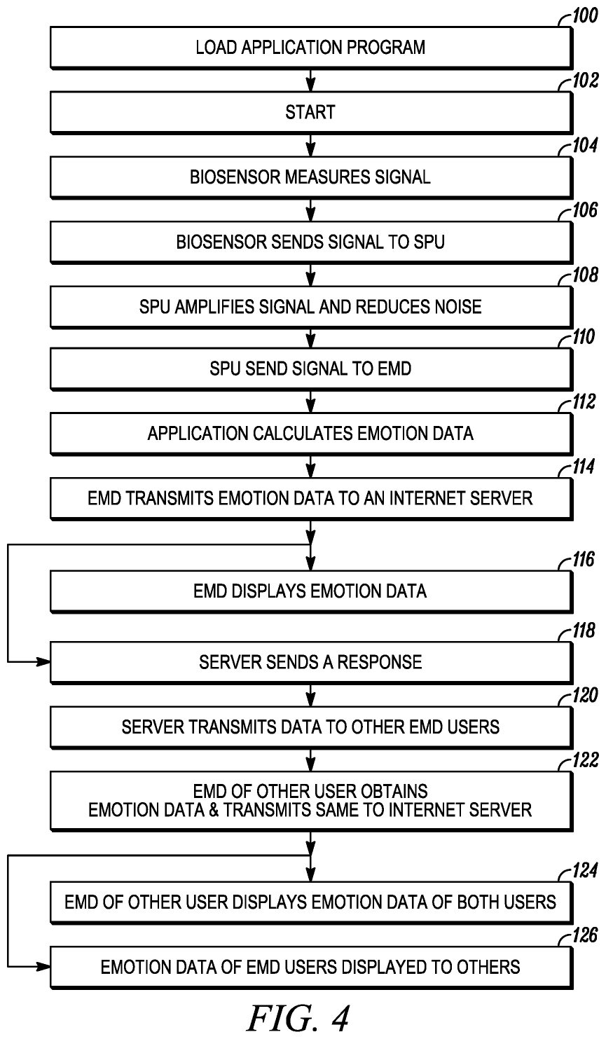

Referring to FIG. 4, a generalized emotion monitoring network is illustrated. A user starts an application program (which in some implementations may constitute a very thin client, while in others may be very substantial) in an EMD (step 102), the application program having been pre-loaded into the EMD or downloaded from the internet (step 100). A biosensor measures a physiological signal (step 104). The biosensor sends the signal to a SPU (step 106) which amplifies the signal and reduces artifact and noise in the signal (step 108). For some types of biosensors, e.g. camera or microphone, this step may be omitted and the signals are processed in a later step to remove artifact and noise, e.g., by discarding signal epochs with poor image or audio quality, and data outliers. The SPU transmits the processed signal via a wired or wireless connection to the EMD (step 110). The EMD further processes the signal and calculates a variety of emotion related data, such as emotional arousal and valence measures (step 112). The EMD displays the emotion data to the user (step 116) and transmits the emotion data to an internet server via a telecommunications network (step 114). An application program resident on the internet server processes the emotion data and sends a response to the user (step 118). It should be noted that the application program may reside on one or more servers or cloud infrastructure connected to the internet and the term “response” here is used generally.

Depending on implementation, the internet server may then transmit the emotion data to one or more remote users equipped with an EMD (step 120) where the emotion data are displayed (step 124). The remote user's EMD similarly calculates their emotion data from physiological signals and transmits it to an internet server to be shared with other users (step 122). The emotion data of all the EMD users may be displayed for review by others on the network and stored for asynchronous review and trend analysis of results of similar interactions over time (step 126).

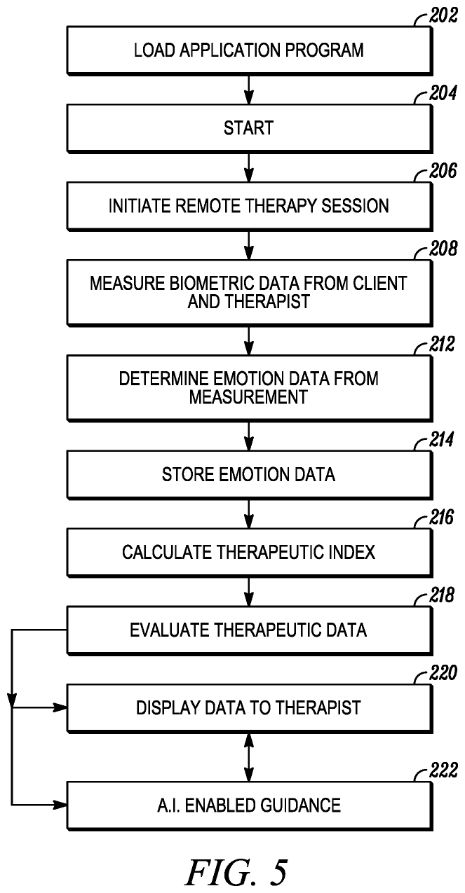

In an implementation for emotion monitoring during teletherapy, and referring to FIG. 5, a first step in a method according to present principles is to load an application program into an EMD for a client and for a therapist (step 202). The application programs may be downloaded from the internet or pre-loaded prior to beginning teletherapy. The client and the therapist start their application programs (step 204). The therapist and client initiate a video call for a remote therapy session (step 206). The EMDs of the therapist and patient may be coupled by way of a video and/or voice channel for communication, or other devices used. Physiological signals related to emotion are monitored by biosensors during the teletherapy session from the client and from the therapist (step 208). Emotion data for the client and the therapist are then derived from the physiological signals utilizing techniques described above (step 212). The emotion data may include emotion arousal and valence components, or other such emotion data. The biometric data and/or the emotion data may then be stored together with clinical notations and other such information for later review (step 214). In some implementations the emotion data may be embodied by an emotional profile, corresponding to client's responses to various standardized stimuli.

A variety of other steps may then be taken depending on implementation. The emotion data of the client and therapist may be compared to assess their working relationship, including calculating an index of therapeutic alliance (step 216). The emotion data and therapeutic alliance may also be compared with others, either individually or within an aggregate (step 218), such as for evaluating patient outcomes across therapy sessions, and to develop predictive analytics. The physiological signals, emotion data, and therapeutic alliance index may be displayed on the EMD of the therapist, or on another device, e.g. in the form of a dashboard to provide real-time feedback and clinical information during the teletherapy session (step 220). In some implementations, a virtual assistant, expert system, or other artificial intelligence application may monitor the therapeutic interaction, including the semantic content and biometric data, to guide the therapist based on subtle patterns that the therapist might otherwise miss (step 222).

It will be understood that the above description of the apparatus and method has been with respect to particular embodiments of the invention. While this description is fully capable of attaining the objects of the invention, it is understood that the same is merely representative of the broad scope of the invention envisioned, and that numerous variations of the above embodiments may be known or may become known or are obvious or may become obvious to one of ordinary skill in the art, and these variations are fully within the broad scope of the invention. For example, while certain wireless technologies have been described herein, other such wireless technologies may also be employed. In another variation that may be employed in some implementations of the invention, the measured emotion data may be cleaned of any metadata that may identify the source. Such cleaning may occur at the level of the mobile device or at the level of the secure server receiving the measured data. In addition, it should be noted that while implementations of the invention have been described with respect to sharing emotion data over the internet, the invention also encompasses systems in which such sharing is performed by other means. Accordingly, the scope of the invention is to be limited only by the claims appended hereto, and equivalents thereof. In these claims, a reference to an element in the singular is not intended to mean “one and only one” unless explicitly stated. Rather, the same is intended to mean “one or more”. All structural and functional equivalents to the elements of the above-described preferred embodiment that are known or later come to be known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present invention is intended to be dedicated to the public regardless of whether the element, component, or method step is explicitly recited in the claims.

1. A method for monitoring emotion data in a network using a mobile phone, comprising:
instantiating an application on a mobile phone, the application downloaded from an internet server, the application configured to cause the mobile phone to receive a physiological signal from a biosensor and process the signal to reduce the presence of artifacts;
running the application in the mobile phone;
receiving physiological signals from the biosensor;
computing emotion data from the received physiological signals using the downloaded application, the computed emotion data including a component of emotional arousal or a component of emotional valence or both;
displaying the computed emotion data using the downloaded application;
transmitting the emotion data to an internet server using a wireless network;
receiving a response from the internet server; and
displaying the response on the mobile phone on a user interface associated with the downloaded application,
wherein the displayed emotion data provide to a user one or more techniques for learning to control emotions and maintain a healthy mental attitude for stress management, lifestyle, or clinical management.

2. The method of claim 1, further comprising operating the internet server such that the response from the internet server is based on a degree of machine learning performed by the internet server, wherein the machine learning is trained on the received physiological signals and the computed emotion data.

3. The method of claim 1, wherein the biosensor is a sensor monitoring skin conductance heart rate, or body heat signatures, a camera monitoring facial expressions wherein the emotion data is derived from the facial expressions, a microphone monitoring a voice signal, wherein the emotion data are derived from voice features, or a combination of the above.

4. The method of claim 1, wherein the signals received from the biosensor are transmitted to an internet server using a wireless network and the computing of emotion data is performed by instructions residing in non-transitory computer medium on the server.

5. The method of claim 1, wherein a tablet computer, or other mobile computing device, replaces the mobile phone.

6. A method for monitoring emotion data in a network from a computing device, comprising:
instantiating an application on a computing device, the application downloaded to the computing device;
receiving a signal from a sensor,
determining emotion data from the downloaded application, the emotion data including a component of emotional arousal or a component of emotional valence or both; and
transmitting data corresponding to the received emotion data to one or more other remote computing devices, including data corresponding to the component of emotional arousal or the component of emotional valence, or both;
wherein the computing device is a personal computer, a mobile phone, a tablet computer, a wearable mobile device, or a hardware appliance programmed for this use; and
wherein the remote computing device is configured to receive and cause the display of the emotion data, wherein the displayed emotion data provide an indicator of mental health for clinical management.

7. The method of claim 6, wherein the sensor is a biosensor monitoring skin conductance, heart rate, or body heat signatures, a camera monitoring facial expressions, wherein the emotion data is derived from the facial expressions, a microphone monitoring a voice signal, wherein the emotion data are derived from voice features, or a combination of the above.

8. The method of claim 6, wherein the signals received from the sensor are transmitted to the remote computing device, and wherein the determining of the emotion data from the received signals is performed by an application program running in the remote computing device.

9. The method of claim 6, further comprising receiving other emotion data from the one or more other remote computing devices.

10. The method of claim 6, wherein the indicator of mental health is based on a degree of machine learning, wherein the machine learning is trained on the received signals and or the computed emotion data.

11. The method of claim 6, wherein the computing device and the one or more other remote computing devices are coupled by way of a video or voice communication channel and wherein the emotion data are determined during the duration of the video or video communication.

12. The method of claim 6, wherein the computing device and one or more other remote computing devices are associated with one or more patients and a therapist or other mental health provider.

13. The method of claim 12, wherein the therapist is a virtual therapist or artificial intelligence application.

14. The method of claim 6, wherein the emotion data are stored for asynchronous processing and display on the one or more other remote computing devices.

15. A non-transitory computer-readable medium, comprising instructions for causing a computing device to operate as an emotion monitoring device, the emotion monitoring device connected in a wired or wireless fashion to a sensor, the non-transitory computer readable medium comprising instructions for causing the emotion monitoring device to perform the following steps:
receive signals from a sensor;
compute emotion data from the received signals, the emotion data including a component of emotional arousal or a component of emotional valence or both;
display the emotion data on a user interface of the emotion monitoring device;
transmit the emotion data to an internet server using a wireless network;
receive a response from the internet server; and
display the response on the user interface of the emotion monitoring device,
wherein the computing device is a personal computer, a tablet computer, a mobile phone, a wearable device, or a hardware appliance programmed for this use; and
wherein the displayed emotion data provide an indicator of mental health for clinical management.

16. The medium of claim 15, wherein the internet server is operated such that the response from the internet server is based on a degree of machine learning performed by the internet server, wherein the machine learning is trained on the received signals and the computed emotion data.

17. The medium of claim 15, wherein the sensor is a biosensor monitoring skin conductance, heart rate, or body heat signatures, a camera monitoring facial expressions, wherein the emotion data is derived from the facial expressions, a microphone monitoring a voice signal, wherein the emotion data are derived from voice features, or a combination of the above.

18. The medium of claim 15, wherein the signals received from the sensor are transmitted to the internet server and the computing of emotion data is performed by instructions residing in non-transitory computer medium on the server.

19. A method of monitoring therapeutic alliance of a patient and a therapist during a teletherapy interaction, comprising:
receiving a first signal from a first biosensor monitoring a remote patient during a teletherapy interaction;
receiving a second signal from a second biosensor monitoring a therapist during the teletherapy interaction;
transmitting the first and second signals to an internet server;
deriving emotion data for the patient and for the therapist based on the first and second signals;
calculating a synchrony of the emotion data of the patient and the therapist and basing a calculation of an index of therapeutic alliance on the calculated synchrony;
transmitting and displaying the patient's emotion data and the therapeutic alliance index on a computing device associated with the therapist.

20. A non-transitory computer readable medium, comprising instructions for causing a computing environment to perform the method of claim 19.

21. A system for remote clinical management of mental health comprising:
one or more biosensors;
an application program, residing on non-transitory media in an emotion monitoring device associated with a patient, the emotion monitoring device in communication with an internet server, the application program containing instructions for causing the emotion monitoring device to receive biometric data from the biosensor, and further causing the biometric data to be transmitted to the internet server;
instructions residing on non-transitory media on the internet server for causing the internet server to receive the biometric data and to derive emotion data from the received biometric data, wherein the emotion data includes at least a valence component, the instructions further causing the emotion data to be transmitted to a therapist having an associated computing device;
instructions residing on non-transitory media within the therapist-associated computing device for causing the therapist-associated computing device to receive and display the emotion data, wherein the emotion data provide the therapist with an indicator of the mental health of the patient.

22. A non-transitory computer readable medium for use in a system for managing the mental health of clinical subjects, the system including a computing device connected in a wired or wireless fashion to one or more biosensors, the non-transitory computer readable medium comprising instructions for causing the device to perform the following steps:
receive physiological signals from one or more biosensors monitoring a clinical subject;
compute data associated with emotional responses from the received signals, the emotional responses including a component of emotional arousal or a component of emotional valence or both, wherein the emotional response data provide a measure of a healthy mental attitude;
transmit the emotional response data to a second user system via an internet server connected to a telecommunications network;
receive a response from the second user system;
wherein the computing device is a mobile phone, tablet computer, wearable device, smart display, personal computer, or a hardware appliance programmed for this use; and
wherein the second user system is configured to display the data providing the measure of a healthy mental attitude of the clinical subject.

23. A system for managing mental health of clinical subjects, comprising:
a computing device receiving physiological signals that relate to changes in emotional states of a subject recorded by biosensors;
a means of transmitting the physiological signals to an internet server, or server cloud;
a second computing device for a remote user, the second computing device having a means of receiving of the physiological signals from an internet server or server cloud; an algorithm for processing the physiological signals to derive and display emotional changes of the subject, the emotional changes including a component of emotional arousal or a component of emotional valence, or both; a user interface on the second computing device to display the derived emotional changes, wherein the
the user interface is configured for the remote user to enter information
whereby the remote user may be enabled to assist the subject in learning to maintain a healthy mental attitude for lifestyle and clinical management.

24. A method for monitoring emotion data during a therapeutic interaction between at least three users, comprising:
receiving a first physiological signal from a first biosensor associated with a first user during a therapeutic interaction;
receiving a second physiological signal from a second biosensor associated with a second user during the therapeutic interaction;
deriving first and second emotion data for the first user and for the second user based on the first and second physiological signals;
transmitting and displaying an indicator of the first emotion data to a mobile device associated with the second user; and
transmitting and displaying an indicator of the second emotion data to a mobile device associated with the first user.
transmitting and displaying the derived first and second emotion data to a computing device utilized by a third user, wherein the third user is selected from the group consisting of a marriage counselor, couples therapist, behavioral therapist, psychiatrist, clinician coach, or a virtual counselor, coach or therapist.

25. A non-transitory computer readable medium, comprising instructions for causing a computing device to perform the method of claim 24.
